(19) United States
(12) Patent Application Publication
(10) Pub. No.: US 2005/0148814 A1
(43) Pub. Date: Jul. 7, 2005

(54) Muscle function augmentation

(76) Inventors: Michael C. Fischi, Norcross, GA (US); Nicolas Alexander Fotiou Chronos, Atlanta, GA (US)

Correspondence Address: NEEDLE & ROSENBERG, P.C., SUITE 1000, 999 PEACHTREE STREET, ATLANTA, GA, 30309-3915, US

(21) Appl. No.: 10/751,682

(22) Filed: Jan. 5, 2004

(51) Int. Cl. A61F 13/00; A61B 5/04; A61N 1/362; A61F 2/00

(52) U.S. Cl. 600/37; 600/521

(57) ABSTRACT

Muscle function can be augmented by causing interconnected electrically operated actuators on an external surface of a muscle or organ to compress or contract. The actuators can be arranged in a band, mesh, or other suitable arrangement.

### BACKGROUND

1. Field of the Invention

The present invention relates generally to medical devices and, more specifically, to a medical device capable of augmenting the function of muscle tissue, such as that of a failing heart.

2. Description of the Related Art

In the management of heart failure it oftentimes becomes necessary to consider a heart transplant or the implantation of a mechanical heart assist device. It is well known that there is an inadequate supply of donor hearts to meet demand. Consequently, implantable cardiac assist devices are being utilized as either a bridge to transplant, a bridge to recovery, or for permanent use.

Several forms of cardiac assist devices are known in the art. Some of the known devices include plunger-type and impeller-driven devices for assisting in the movement of blood through the heart. The plunger-type devices involve the diversion of blood from the left ventricle into a cam-driven or pneumatic piston that pumps blood into the aorta. While such devices can effectively augment cardiac output, they are bulky and require a complex surgical implantation into the thoracic and abdominal cavities, respectively. The plunger-type devices are also prone to clot formation with an accompanying high incidence of stroke. The impeller-driven devices utilize an impeller to propel blood from the left ventricle into the aorta. While the known types of impeller-driven devices are less bulky than the plunger-type, they are less effective in augmenting cardiac output and are also prone to clot formation possibly leading to stroke.

Both plunger-type and impeller-driven devices have an inherent predisposition to the formation of blood clots as each requires blood to be shunted through synthetic vasculature or veinous structures, and place the blood in contact with metallic impeller blades or plunger plates.

There is a need for a medical device or system for augmenting or assisting cardiac function that is less prone to blood clot formation and generally less invasive. The present invention addresses this deficiency and others in the manner described below.

### SUMMARY

The present invention relates to an electromechanical apparatus and method useful for augmenting muscle function. The method involves operating electrically operated actuators on an external surface of the muscle or organ.

A method for compressing a bodily organ can comprise the steps of applying a carrier having an electrically operated actuator system to the bodily organ, sensing a pre-determined condition, and in response, operating the actuator system to compress at least a portion of the organ. The carrier comprises electrically operated actuators arranged in a band, mesh, or other suitable arrangement. In some embodiments of the invention, the carrier has a band-like, tubular or cuff-like shape that facilitates the carrier at least partially encircling the organ. In other embodiments of the invention, the carrier has a bag-like, pouch-like or sack-like shape that facilitates the carrier at least partially enveloping the organ. In such embodiments, the carrier is applied to the outside of the organ for the purpose of compressing it. Nevertheless, the carrier can have any other suitable shape and structure. For example, in still other embodiments, the carrier can be applied to the outside of the organ for other purposes, such as attaching it to bones for the purpose of acting as artificial skeletal muscle.

The carrier can, in some embodiments of the invention, compress an organ in synchronization with its native electromechanical cycle. This system may augment the pumping function of a heart, for example, without otherwise interrupting the circulatory system and creating a pro-thrombotic state.

In any given embodiment of the invention, the features described in this patent specification with regard to embodiments of the invention can be included individually or in any suitable combination with each other or with other features. It is to be understood that both the foregoing general description and the following detailed description are exemplary and explanatory only and are not restrictive of the invention, as claimed.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment, and wherein:

FIG. 1 is a generalized perspective view of a system for compressing a portion of a heart in which the carrier has a drawstring fastener;

FIG. 2 is a generalized perspective view of a system for compressing a portion of a heart in which the carrier has a lace-up fastener;

FIG. 3 is a generalized perspective view of the carrier applied to a heart;

FIG. 4 is a generalized perspective view of the carrier compressing the heart;

FIG. 5 is a block diagram of a sensor system that applies signals to the carrier in response to bioelectric signals sensed at an electrode coupleable to the organ;

FIG. 6 illustrates a system in which the carrier is implanted within a human body and inductively coupled to an external power supply;

FIG. 7 is a generalized perspective view of the carrier, partially cut-away to show a mesh-like arrangement of electrically operated actuators;

FIG. 8 illustrates two solenoid-like electrically operated actuators connected in an end-to-end arrangement;

FIG. 9 is a sectional view taken along line 9-9 of FIG. 7;

FIG. 10 an end view of a portion of a solenoid-like electrically operated actuator that can be interconnected with other such actuators along two mutually perpendicular axes to define a mesh;

FIG. 11 is a sectional view of the actuator portion of FIG. 10, taken along line 11-11 of FIG. 10;

FIG. 12 illustrates a portion of a mesh defined by the interconnection of actuators of the type shown in FIG. 10;

FIG. 13 is a generalized perspective view of a system compressing legs;

FIG. 14 is a generalized perspective view of a system compressing a portion of a stomach;

FIG. 15 is a generalized perspective view of a system compressing an esophagus;

FIG. 16 is a generalized perspective view of a system in which an elongate carrier is attached to bone and acts as an artificial skeletal muscle;

FIG. 17 is a generalized perspective view of a carrier having relatively high-powered solenoid-like electrically operated actuators interconnected along two mutually perpendicular axes;

FIG. 18 is a generalized top view of a carrier in which actuators are distributed unevenly around the circumference;

FIG. 19 is a generalized perspective view of a carrier having relatively high-powered solenoid-like electrically operated actuators that constrict straps; and

FIG. 20 is a generalized perspective view of a carrier comprising an electroactive polymer wrap.

### DETAILED DESCRIPTION

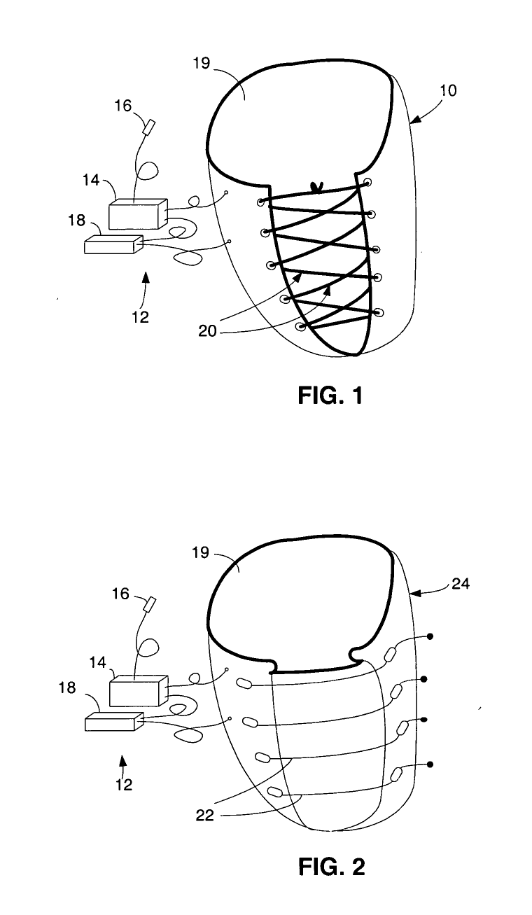

As illustrated in FIG. 1, in an exemplary embodiment of the invention, a system for compressing a bodily organ includes a carrier 10 and a sensor system 12. Although in the illustrated embodiment sensor system 12 is external to carrier 10 and includes a control system 14 with an electrode 16 and a battery or other power supply 18, in other embodiments portions of it can be within carrier 10 or arranged in any other suitable manner with respect to carrier 10. In this embodiment, carrier 10 has a pouch-like, sack-like or bag-like shape with an opening 19 fittable over a portion of an organ. Carrier 10 further has laces 20 for tightening it around the organ. Nevertheless, in other embodiments, any other suitable means for fastening it around the organ or to the organ can be included, such as the drawstring or zip-tie fasteners 22 shown on a carrier 24 in FIG. 2.

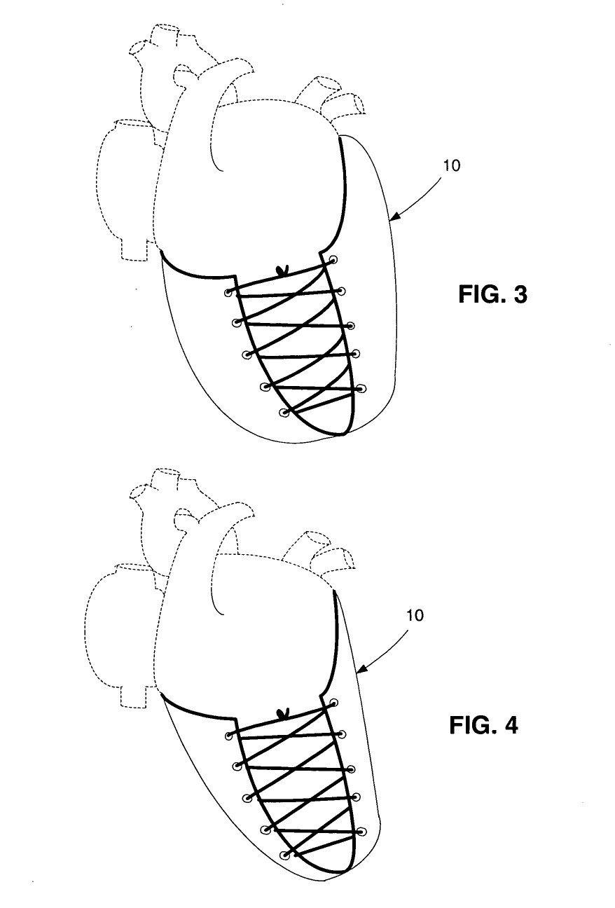

As illustrated in FIG. 3, carrier 10 is applied to a heart. It can be fitted so that it partially envelopes the ventricular portion of the heart, with the closed end of carrier 10 at the apex, and the rim of opening 19 at or near the atrio-ventricular groove. In FIG. 4, though none of the figures are drawn to scale, carrier 10 is illustrated as compressing or squeezing the portions of the heart to which it is applied. Thus, in this embodiment of the invention, carrier 10 is roughly on the order of a human heart in size. As described in further detail below, compression can occur evenly throughout carrier 10, thereby compressing the entire ventricle, or, in other embodiments of the invention, compression can occur at successive portions of carrier 10 in an undulating, peristaltic or other non-homogeneous manner. For example, carrier 10 can initiate compression at the apex and move the area of compression upwardly toward the atrioventricular groove to mirror the heart's natural pattern of contraction as much as possible.

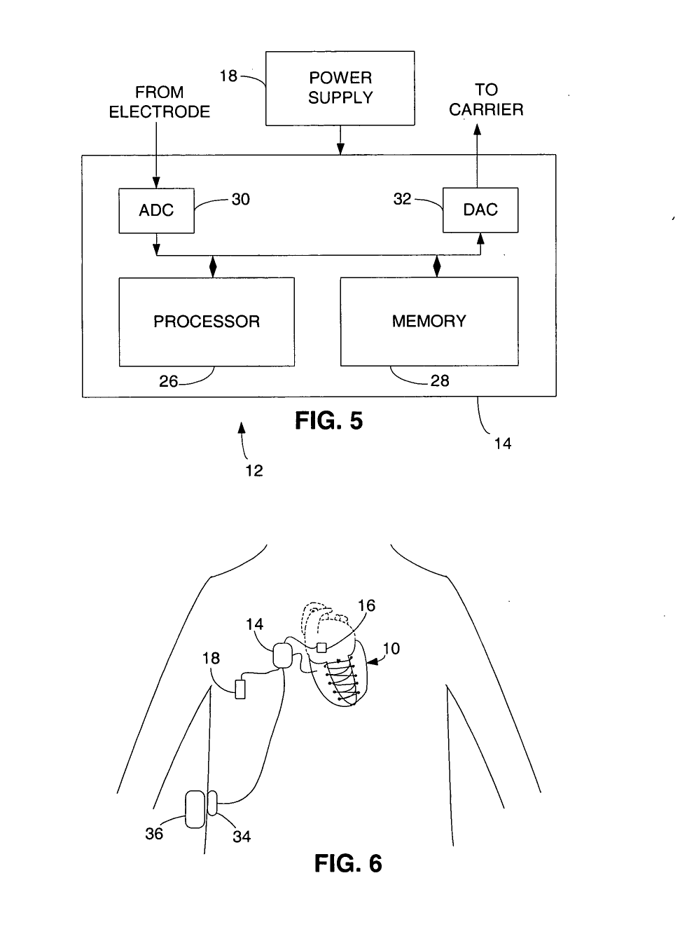

As further illustrated in FIG. 5, sensor system 12 includes control system 14 and power supply 18. Control system 14 includes a suitable processor system 26, such as one having a microprocessor or microcontroller chip, as well as other hardware, software and firmware elements that persons skilled in the art understand are included in implantable intelligent biomedical devices, such as suitable read-only and random-access memory 28, an analog-to-digital conversion (ADC) system 30, and a digital-to-analog conversion (DAC) system 32. ADC system 30 provides processor system 26 with digital signals corresponding to the analog bioelectric signals picked up from the organ by electrode 16 (FIG. 1). DAC system 32 provides carrier 10 with analog signals that cause it to compress and relax in response to the digital output signals computed by processor system 26. Although the compression process or method is controlled by processor system 26 under the control of suitable programming embodied in software or firmware, in other embodiments of the invention it can be controlled by any suitable type of logic or combinations of types, including programmable logic (such as that of processor system 26) and fixed logic that can be respectively programmed or adapted to effect the process or method described in this patent specification. In the illustrated embodiment, the programming for processor system 26 is embodied as software or firmware stored in a non-volatile read-only memory (ROM) portion of memory 28. Working memory (i.e., RAM) for facilitating the computations performed by processor system 26 and related purposes can also be included in memory 28 and, additionally or alternatively, within processor system 26 itself.

Sensor system 12 can be miniaturized and sealed in a biologically inert housing suitable for implantation in the body along with carrier 10. Electrode 16 (FIGS. 1-2) can be implanted on the heart in the manner of a conventional epicardial lead to pick up the R-wave produced by the heart. As described below in further detail, in response to sensing each R-wave, processor system 26 produces a signal that causes carrier 10 to begin compressing. Processor system 26 also causes causes information descriptive of the R-wave to be stored in a suitable area of memory 28. Processor system 26 can time the interval between R-waves, which represents the interval between contraction during systole and release during diastole, store the interval time in memory 28, and maintain a moving average of such interval times. When processor system 26 determines that an amount of time equal to the moving average has elapsed since causing carrier 10 to begin compressing, processor system 26 alters the signal provided to carrier 10, e.g., by reducing its voltage or current, reversing its polarity or other suitable change, in a manner that causes carrier 10 to cease compressing. In another embodiment of the invention (not shown), utilization of a latching-type solenoid (magnetically charged core) permits reversal of the polarity, which will actively cause the carrier to expand rather than merely allow carrier 10 to passively relax back into the expanded state (see FIG. 3). In still another embodiment (not shown), the device may utilize flat, rectangular-shaped solenoids or solenoids or any other suitable shape in addition to or in place of the more common cylindrical ones. By utilizing a flat, rectangular solenoid casing with a flat, rectangular core that slides in and out, the same power may be achieved as with a cylindrical solenoid of similar mass and number of coils, but in a more space-effective configuration (thus more easily fitting in the chest cavity).

Although in the exemplary embodiment of the invention, the R-wave sensed by electrode 16 triggers processor system 26 to operate carrier 10, in other embodiments other suitable devices (not shown) can sense other conditions known to reflect the cardiac cycle, such as a change in arterial pressure, to trigger operation of carrier 10.

As illustrated in FIG. 6, carrier 10 is implanted within a human body and applied to the heart as described above. Power supply 18, which can comprise a suitable battery within a biologicaly inert housing, is supplemented with an inductive power receiver 34 that is also implanted within the body just beneath the skin. In operation, power receiver 34 is inductively coupled to power transmitter 36, which can comprise a suitable battery and inductive coil and associated circuitry, worn on the person's belt or otherwise carried externally to the body. Power receiver 34 thus receives power transcutaneously. In such embodiments, power supply 18 can serve as a back-up in case this transcutaneous inductive power supply system malfunctions or otherwise fails to adequately power carrier 10 and sensor system 12. In other embodiments, power receiver 34 and power transmitter 36 can be included instead of power supply 18.

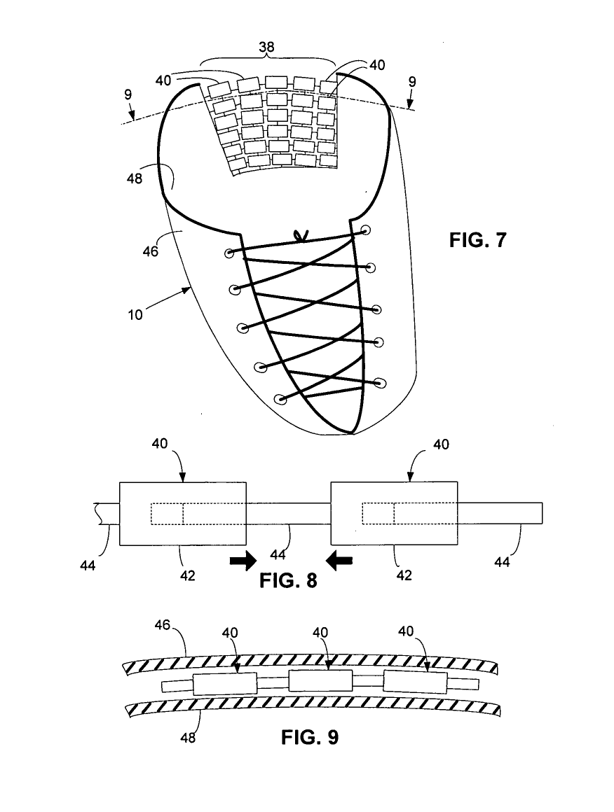

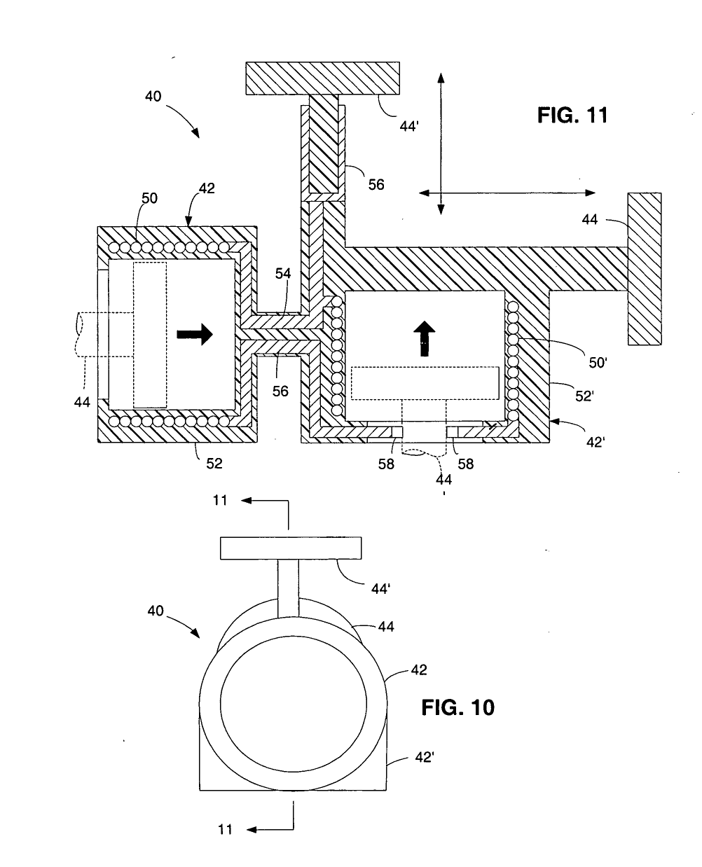

As illustrated in FIG. 7, carrier 10 comprises an electrically operated actuator system 38. Actuator system 38 comprises a plurality of mutually interconnected electrically operated actuators 40. In the illustrated embodiment, actuators 40 are interconnected along two generally orthogonal axes in a mesh-like or grid-like arrangement. Note that the arrangement is depicted in a generalized or conceptualized form in FIG. 7 for purposes of clarity, and described in further detail below with regard to FIGS. 10-12.

Actuators 40 operate generally in the manner of a solenoid, which, as well-known in the art, is a device that converts electrical current into an axial force using an energized wire coil to attract (or, equivalently, repel) a permanent magnet or similar magnetized member along the central axis about which the coil is wound. As such, as illustrated in FIG. 8, each actuator 40 can be described generally or conceptually as having (at least one) coil portion 42 and (at least one) magnetic member 44. Magnetic member 44 moves axially within coil portion 42 when coil portion 42 is energized, thereby contracting the adjacent actuators 40 together in the directions of the arrows. It is such a contractile force that contributes to the compression of carrier 10, as described in further detail below.

Referring again to FIGS. 7-9, in the exemplary embodiment, carrier 10 comprises actuators 40 arranged in an end-to-end manner generally along a (first) axis to define a band-like arrangement of actuators 40, as best seen in FIGS. 8-9. As noted above, bands can further be arranged laterally adjacently to one another or stacked on top of one another generally along another (second) axis, such that the result is a mesh-like or grid-like arrangement or array of actuators 40. Laterally disposed actuators 40 can be interconnected, as in the exemplary embodiment, or independent of one another. Note that reference is made to such a “first axis” and “second axis” only for illustrative purposes, and that such axes are not actually linear in this embodiment of the invention because together they define not a plane but rather portions of the two-dimensionally curved surface or wall that characterizes the bag-like, sack-like or pouch-like shape of carrier 10 that enables it to at least partially envelope the portion of the organ to which it is applied.

Sensor system 26 provides the signals that energize actuators 40. As described above, sensor system 26 can energize all actuators 40 of carrier 10 together in synchronism or, in other embodiments, can energize selected subsets of them. For example, by suitably programming sensor system 26, successive bands of actuators 40 can be energized to provide the undulating compressive force described above. By providing suitably selective signals between sensor system 26 and carrier 10, e.g., by providing multiple signal lines or by multiplexing or encoding the signals and providing suitable demultiplexing or decoding circuitry (not shown) within carrier 10, sensor system 26 can control actuators 40 in groups or subsets of any suitable number, even to the extent of controlling each actuator 40 individually. In such a manner, carrier 10 can define an active surface that sensor system 26 can cause to assume various shapes to more closely mirror the heart's natural pattern of contraction or provide other unique compression patterns.

In some embodiments of the invention, carrier 10 can further include coverings over one or both surfaces defined by the mesh, band or other group of actuators 40 of an actuator system. As illustrated in FIGS. 7 and 9, there can be an outer covering 46 over the outer or exterior surface or wall of the actuator system and an inner covering 48 over the inner or interior surface or wall of the actuator system, with the mesh of actuators 40 sandwiched between coverings 46 and 48. Coverings 46 and 48 protect the heart against friction injury from the actuator system. The space between coverings 46 and 48 can be filled with a suitable electrically and biologically inert material, such as silicone, with the mesh of actuators 40 thus embedded or floating in the silicone. Coverings 46 and 48 can each comprise, for example, a flexible membrane made of an electrically insulating, biologically inert material, such as rubber. In other embodiments, a covering can be made of any other suitable material and can comprise more than the one layer shown. The electrically insulative property of such material insulates the actuator system from the native electrical activity of the heart, and insulates the heart from electrical signals present in the actuator system. Either or both of coverings 46 and 48 can be coated with an anti-fibrotic drug, such as rapamycin, phosphorylcholine or paclitaxel. Such drugs can be applied in combination with a drug elivery polymer. Coverings 46 and 48 can be coated with a silicone or TEFLON®-based compound to reduce friction with the cardiac surface during operation.

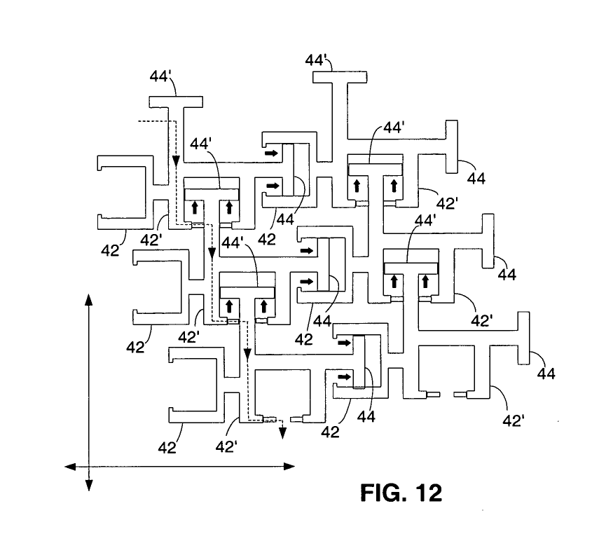

In the exemplary embodiment, in which actuators 40 are arranged in a mesh-like manner along two orthogonal axes, each actuator 40 can have, for example, the two-axis solenoid-like structure illustrated in FIGS. 10-12. Two coil portions 42 and 42′ are oriented along mutally orthogonal axes. Two magnetized members 44 and 44′ are also oriented along mutually orthogonal axes. As illustrated in FIG. 12, the mesh-like or array-like structure results from magnetized member 44 of one actuator 40 being reciprocatingly disposed in a piston-like manner within cylinder-like coil portion 42 of an adjacent actuator 40, and magnetized member 44′ of one actuator 40 being reciprocatingly disposed in a piston-like manner within cylinder-like coil portion 42′ of an adjacent actuator 40. This mesh-like actuator system can be constructed using microelectromechanical methods that are well-known in the art and within the capabilities of persons skilled in the art. For example, known microelectromechanical structures (MEMS) technology can be employed. Nevertheless, the size of the actuator system as a whole and the size and number of its individual actuators 40 can be scaled to correspond with the intended use. For example, in the illustrated embodiment of the invention, in which carrier 10 is used to augment or assist cardiac function, the actuator system mesh can consist of, for example, several hundred actuators 40, each on the order of one cubic centimeter in size.

Referring to FIG. 11, coil portion 42 comprises a miniature wire coil 50 (not to scale) embedded or otherwise mounted within a supporting cylinder structure 52, and coil portion 42′ comprises a miniature wire coil 50′ (not to scale) embedded or otherwise mounted within a supporting cylinder structure 52′. A conductor 54, also embedded in or otherwise mounted within the supporting structure of actuator 40 carries current from a cylindrical collector ring or bushing 56, through wire coil 50, through a conductor 56, and to a pair of brushes 58. Brushes 58 contact bushing 56 of an adjacent actuator 40, as illustrated in FIG. 12. The dashed line in FIG. 12 indicates the current carried through a chain of adjacent actuators 40 in this manner, thereby avoiding the need to couple each actuator 40 to an individual conductor or power buss.

In operation, in essentially the manner of a conventional solenoid, currents in wire coils 50 and 50′ create magnetic fields and corresponding forces upon magnetized members 44 and 44′. The forces urge magnetized members 44 and 44′ in the directions indicated by the respective arrows in FIGS. 11 and 12. It can readily be seen in FIG. 12 that the resulting movement contracts the mesh of actuators 40, causing carrier 10 to compress the organ to which it is applied (see FIG. 4). When the current is removed, the mesh expands again. The expansion can, in some embodiments of the invention, occur passively as, for example, blood entering the ventricle expands carrier 10 back to a relaxed state. Nevertheless, in other embodiments, current can be applied in the opposite direction to create opposite magnetic fields, thereby drawing magnetized members 44 and 44′ in directions opposite those indicated by the arrows in FIGS. 11 and 12 and actively expanding carrier 10. In other words, alternating the direction of the current in such embodiments causes carrier 10 to alternately contract and expand.

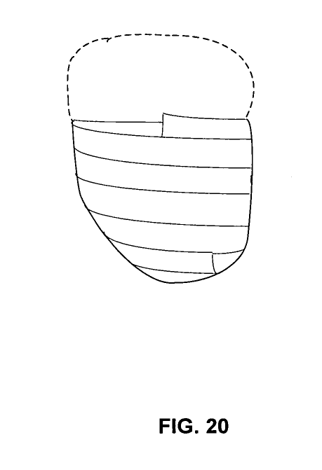

Although in the illustrated embodiment of the invention, actuators 40 have a two-axis structure that facilitates arranging them in the above-described mesh-like manner along two generally orthogonal axes, in other embodiments they can have other structures, such as those with two non-orthogonal axes, that facilitate other arrangements. The arrangement can define a mesh, as in the above-described embodiment, or can define a band, cylinder or other suitable shape, as described below. Furthermore, although in the illustrated embodiment, actuators 40 are solenoid-like devices that contract (or, equivalently, extend) in response to current-induced magnetic fields, in other embodiments they can employ other suitable electrically operated technologies, such as utilization of an electroactive, conductive polymer wrap as shown in FIG. 20. Such wrap material is well-known in the art, and therefore its structural details are not described here. The conductive polymer has the capability to constrict when a voltage is placed across it, thus squeezing the heart during systole. The spiral wound polymer strip(s) contracts down like a wound coil tightening around the heart, thus facilitating its pumping action. When the voltage is removed (during diastole), the conductive polymer will relax and the coil unwinds releasing the pressure. The action of alternately contricting and relaxing the conductive polymer spiral that is wrapped around the heart is enacted in sych with the hearts mechanical systole and diastole.

Although suitable conductive polymer wrap material is readily available from a variety of commercial sources, it may be instructive to note that, on a molecular level, it comprises a conjugated system whereby single and double bonds alternate along the polymer chain. The neutral polymer chain is then subjected to partial oxidation thus generating polyanions. At the same time, anions are inserted to neutralize the positive charges in the polymer. This process results in a polymer capable of a reversible oxidative/reductive (i.e. redox) process. The conductive polymer film is sandwiched in a bi-layer or tri-layer with an adhesive polymer film or “non-volume changing” film. During the redox reaction that occurs when a voltage is placed across the conductive polymer, anions or cations present in the “non-volume changing” film shift into the conductive polymer causing the conductive polymer to increase in molecular volume, swell, and then bend. An example of such a compound would be two polyanaline films (conductive polymer) separated by an HCl impregnated adhesive film (to supply the cations).

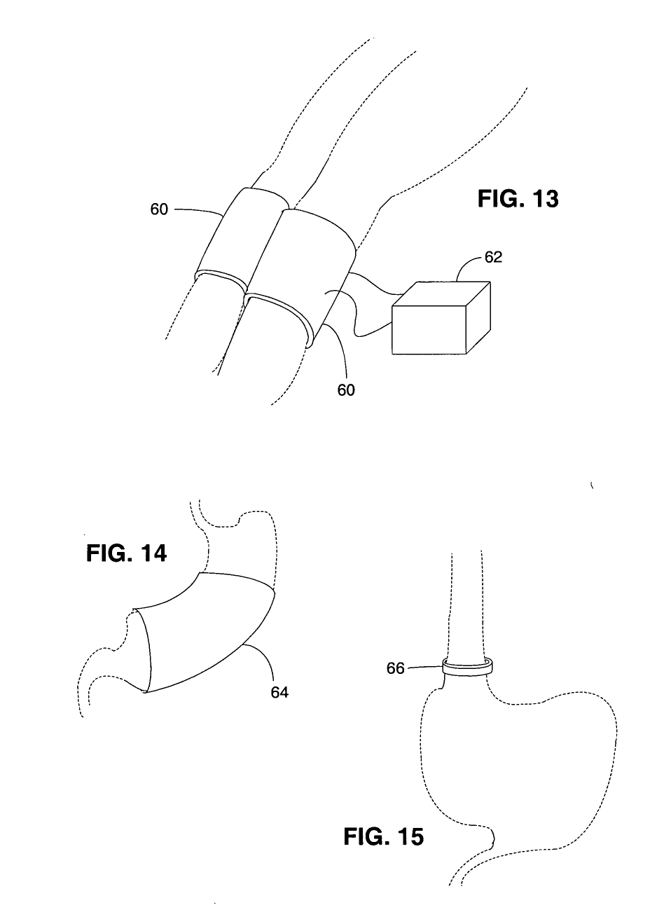

As illustrated in FIGS. 13-15, in other embodiments of the invention, organs or portions of the body other than a heart can be compressed for various purposes. For example, as illustrated in FIG. 13, a device comprising tubular, band-like or cuff-like carriers 60 and a sensor system 62 is similar to that described above with regard to FIGS. 1-12, but in this embodiment of the invention their shape facilitates applying carriers 60 to a person's extremities. Although not shown for purposes of clarity, each of carriers 60 includes a suitable fastener, such as laces, for securing the band edges (also not shown) to each other around the leg.

Applying carrier 60 to a person's calf, the device can be used to prevent deep venous thrombosis (DVT). In the manner of conventional pneumatic devices used for this purpose, carrier 60 compresses the calf intermittently to reduce stasis and improve venous return from the lower extremities. Sensor system 62 can readily be programmed to time a suitable interval at which to causes carrier 60 to compress.

Similarly, a pair of carriers 60 (not shown) can be applied to each leg for enhanced external counterpulsation (EECP). In the manner of conventional pneumatic devices used for EECP, a pair of carriers 60 operate in sequence in a peristaltic or “milking” action to pump blood upward towards the heart. Sensor system 62 can readily be programmed to compress at the end of each detected heartbeat and relax just as the next heartbeat begins.

Carriers 60 can comprise a mesh-like arrangement of actuators 40 as described above with regard to other embodiments of the invention. Such a tubular, band-like or cuff-like carrier 60 can consist of as few as a single band of actuators 40 (i.e., a plurality of actuators 40 arranged generally linearly or end-to-end) or can comprise many bands arranged laterally adjacent to one another in a mesh (see, e.g., FIG. 7).

In tubular embodiments in which actuators 40 are arranged along two axes to define a mesh, it can be noted, as with the other embodiments described above, that when the carrier assumes its tubular shape, one “axis” is actually more circular than a linear axis due to the substantially cylindrical, tubular shape of carrier 60 that enables it to at least partially encircle the organ to which it is applied. (The other axis is substantially linear and defines the cylindrical central axis along which carrier 60 is elongated.) Nevertheless, the term “axis” is used in this patent specification for convenience and because it is descriptive of the actuator system when laid flat. Note, for example, that carrier 60 and other such tubular carriers of the present invention can be flexible enough that they can lay flat or assume a rectangular, planar shape when not wrapped around a leg or other organ. An actuator system in a flat state and the two orthogonal axes can be clearly seen in FIG. 12.

As illustrated in FIG. 14, in an embodiment of the device similar to that described above with regard to FIG. 13, a carrier 64 is applied to a stomach to assist gastrointestinal peristalsis. Under control of a suitable sensor system (not shown for purposes of clarity), carrier 64 squeezes or compresses the stomach in the presence of a food bolus. The sensor system can operate in response to a signal sensed by a strain gauge or other suitable transducer (not shown) mounted on the stomach wall or in response to a signal the patient initiates by, for example, triggering a remote control unit (not shown).

As illustrated in FIG. 15, in an embodiment of the device similar to that described above with regard to FIGS. 13-14, a carrier 66 is applied to a person's lower esophageal sphincter to treat achalasia, which is a disorder of esophageal motility characterized by the absence of peristalsis, an elevated pressure of the lower esophageal sphincter, and the failure of the lower esophageal sphincter to relax during swallowing. Carrier 66 can serve as an artificial sphincter. Under control of a suitable sensor system (not shown for purposes of clarity), carrier 66 squeezes or compresses to close off the esophagus in response to an increase in pressure in the esophagus, opens for a predetermined time interval thereafter, and then compresses again to close off the esophagus when the pressure is relieved. Similar embodiments of the device can also be used as replacements or to augment the function of other anatomical sphincters, such as the anal sphincter of a patient requiring reconstructive surgery for anal cancer.

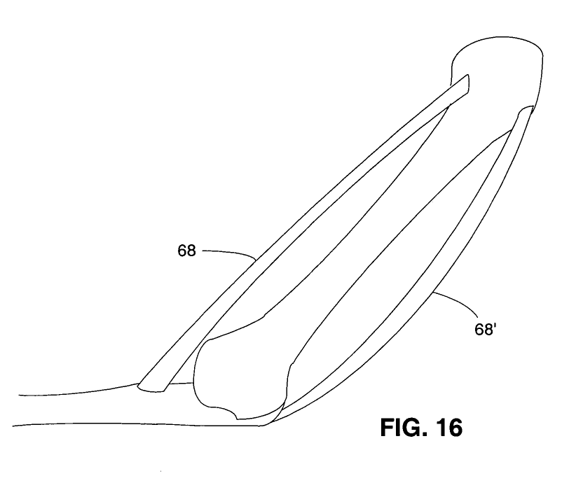

As illustrated in FIG. 16, in another embodiment of the device, an elongated, band-like carrier 68 is attached at its opposing ends to points on bones to act as an artificial skeletal muscle. Carrier 68 can have an actuator system like that described above with regard to other embodiments. In other words, carrier 68 can comprise a single band of actuators 40 (i.e., a plurality of actuators 40 arranged generally linearly or end-to-end) or a mesh-like arrangement in which several such bands are laterally adjacent to one another, with the ends of each band attached to the bone. Under control of a suitable sensor system (not shown for purposes of clarity), such as one that senses nervous or muscular signals, carrier 68 contracts or compresses, thereby drawing closer together the points to which its ends are attached and causing the joint to flex. The sensor system can operate in response to a signal sensed by a strain gauge or other suitable transducer (not shown) mounted on the stomach wall or in response to a signal the patient initiates by, for example, triggering a remote control unit (not shown). Opposing carriers 68 and 68′ can be applied to emulate opposing muscles, such as biceps and triceps, respectively. Also, in similar embodiments, the ends can be attached to structures other than bones, such as opposed cups (not shown) between which a portion of an organ or other object can be squeezed. The structures can be essentially anything having points on them from which a force can be exerted on an object between the points or to which the points are attached.

A sheet-like carrier defined by an arrangement with many adjacent actuator bands can be used to replace or augment other types of muscle. As noted above, tubular carriers 60 and 64 essentially have sheet-like shapes with opposing ends connectable or fastenable together to define what may variously be referred to as a tube, cylinder, ring, cuff, etc. In general, an actuator system can define a sheet-like material that can be used to construct carriers of any suitable shape with any suitable contours and other features. As FIG. 12 illustrates, a flat, mesh-like actuator system with sufficient flexibility bears some resemblence to chainmail material, and can be used as a material for constructing the above-described carriers and other articles.

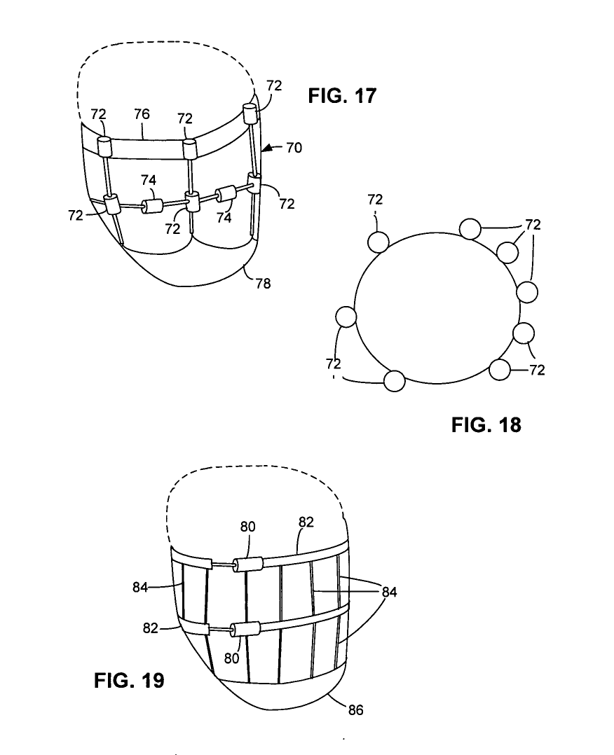

As illustrated in FIG. 17, in an embodiment of the device similar to those described above with regard to, for example, FIGS. 1-6, a carrier 70 has solenoid-like electrically operated actuators 72 along a longitudinally axis that are interconnected with similar actuators 74 along a circumferential axis. A strap 76 at one (longitudinal) end of carrier 70 can be used to secure it to the atrioventricular groove of a heart (indicated generally in dashed line). A parachute-like or cup-like endpiece 78 at other end of carrier, made of a suitably flexible material such as rubber or fabric, encloses the apex of the heart. Note that there are fewer of actuators 72 and 74 than in the above-described embodiments, but actuators 72 and 74 are larger and more powerful. Nevertheless, a strap 76 or similar element, a cup-like endpiece 78 or similar element and other elements of the embodiment illustrated in FIG. 17 can be included in the above-described embodiments of the invention in which the actuators are smaller and may define a mesh-like structure. Still other embodiments can have only longitudinal actuators 72 and not circumferential actuators 74, or, alternatively, only circumferential actuators 74 and not longitudinal actuators 72, as indicated in FIG. 19. In the embodiment illustrated FIG. 19, circumferential actuators 80 constrict straps 82 to compress the heart as described above with regard to other embodiments. Longitudinal struts 84 extend between cup-like endpiece 86 and straps 82, between endpiece 86 and actuators 80, and between the plurality of straps 82 to add structural integrity.

FIG. 18 is a generalized top view of a carrier (e.g., that illustrated in FIG. 17) in which actuators 72 are distributed unevenly around the circumference, illustrating the point that in any of the above-described embodiments, the actuators need not be distributed in a uniform manner or pattern. For example, as illustrated in FIG. 19, carrier 70 can be applied to a heart such that the number of actuators 72 on the left ventricular side is greater than the number of actuators 72′ on the right ventricular side, thereby tending to bias the squeezing or compressive force toward the left side ventricular side. As noted above with regard to other embodiments, biasing the compressive force or otherwise causing uneven compression or a pattern of compression can also be achieved by electronic means by applying appropriately proportioned or sequenced control signals to the actuators.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for compressing a bodily organ, comprising the steps of:
applying a carrier comprising an electrically operated actuator system to the bodily organ;
sensing a pre-determined condition; and
operating the actuator system in response to sensing the pre-determined condition while the carrier is applied to the organ to compress at least a portion of the organ.

2. The method of claim 1, wherein the step of applying a carrier comprises surgically implanting the carrier in an animal body.

3. The method of claim 1, wherein the step of applying a carrier comprises at least partially encircling the organ with the carrier.

4. The method of claim 1, wherein the step of applying a carrier comprises at least partially enveloping the organ with the carrier.

5. The method of claim 1, wherein the step of applying a carrier comprises attaching a portion of the carrier to the organ.

6. The method of claim 5, wherein the step of attaching a portion of the carrier to the organ comprises attaching first and second opposing ends of a generally elongate carrier to first and second tissue sites.

7. The method of claim 1, wherein the step of applying a carrier comprises applying a carrier to muscle.

8. The method of claim 1, wherein the step of applying a carrier comprises applying a carrier to a stomach.

9. The method of claim 1, wherein the step of applying a carrier comprises applying a carrier to a heart.

10. The method of claim 9, wherein the step of applying a carrier comprises applying a carrier at an atrioventricular groove region of the heart.

11. The method of claim 10, wherein the step of applying a carrier further comprises the step of fastening a band around the atrioventricular groove region to secure the carrier to the heart.

12. The method of claim 1, wherein the step of applying a carrier comprises securing a fastener.

13. The method of claim 12, wherein the step of securing a fastener comprises fastening laces of the carrier.

14. The method of claim 1, wherein the step of operating the actuator system in response to the pre-determined condition comprises applying an electrical signal to a solenoid-like contractile actuator.

15. The method of claim 14, wherein the step of sensing a pre-determined condition comprises sensing an R-wave.

16. The method of claim 1, wherein the step of applying a carrier comprises applying a carrier having a plurality of electrically operated actuators arranged linearly.

17. The method of claim 16, wherein the step of applying a carrier comprises applying a band comprising a plurality of electrically operated actuators at least partially encircling the organ.

18. The method of claim 1, wherein the step of applying a carrier comprises applying a carrier having a plurality of electrically operated actuators arranged in a mesh.

19. The method of claim 1, wherein the step of applying a carrier comprises applying a mesh comprising a plurality of electrically operated actuators at least partially enveloping the organ.

20. A device for assisting a heart, comprising:
a sensor system responsive to a train of bioelectric signals; and
a carrier having an electrically operated actuator system comprising a plurality of mutually interconnected electrically operated actuators, the electrically operated actuator system responsive to the sensor system by compressing at least a portion of the heart at intervals determined in response to the bioelectric signals.

21. The device of claim 20, wherein the bioelectric signals comprise an R-wave.

22. The device of claim 20, wherein the sensor system comprises:
an electrode coupleable to the heart; and
a processor system programmed or adapted to compute the intervals in response to the bioelectric signals.

23. The device of claim 20, wherein the sensor system comprises a transcutaneous inductive power supply coupler.

24. The device of claim 20, wherein the electrically operated actuators are arranged linearly end-to-end.

25. The device of claim 24, wherein each electrically operated actuator is a solenoid-like contractile actuator.

26. The device of claim 25, further comprising a strap connected to at least some of the actuators.

27. The device of claim 25, further comprising a cup-like endpiece interconnecting at least some of the actuators.

28. The device of claim 20, wherein the electrically operated actuators define a mesh.

29. The device of claim 28, wherein the mesh has a bag-like shape with an opening fittable over the heart.

30. The device of claim 29, further comprising a fastener.

31. The device of claim 30, wherein the fastener comprises laces.

32. The device of claim 28, wherein each electrically operated actuator is a solenoid-like contractile actuator.

33. The device of claim 32, wherein the mesh comprises a plurality of interconnected links, each link comprising a first coil, a second coil, a first magnetic piston and a second magnetic piston, the first coil and second coil are arranged in fixed relation to each other along mutually perpendicular first and second axes, and the first magnetic piston and second magnetic piston are arranged in fixed relation to each other along the first and second axes, and wherein a magnetic piston of a link is received within a coil of an adjacent link.

34. The device of claim 33, wherein each piston includes means for carrying current and transferring current from a link to an adjacent link.

35. The device of claim 28, wherein the carrier comprises an electrically insulating biologically inert membrane covering at least a portion of a surface of the mesh.

36. The device of claim 35, wherein the membrane comprises rubber.

37. The device of claim 28, wherein the carrier comprises two flexible membrane layers sandwiching the mesh.

38. The device of claim 37, wherein the flexible membrane layers comprise rubber.

39. The device of claim 37, wherein the carrier comprises a silicone lubricant coating between the two flexible membrane layers.

40. The device of claim 28, wherein the carrier is coated with an anti-fibrotic drug.

41. The device of claim 40, wherein the coating is rapamycin, phosphorylcholine or paclitaxel.

42. A device for assisting a heart, comprising:
(a) a sensor system, comprising:
(i) an electrode coupleable to the heart; and
(ii) a processor system programmed or adapted to compute trigger intervals in response to R-waves received by the electrode; and
(b) a carrier, comprising:
(i) an electrically operated actuator system having a plurality of mutually interconnected electrically operated actuators defining a mesh, the electrically operated actuator system responsive to the sensor system by compressing at least a portion of the heart at the trigger intervals, the mesh comprising a plurality of interconnected links, each link comprising a first coil, a second coil, a first magnetic piston and a second magnetic piston, the first coil and second coil arranged in fixed relation to each other along mutually perpendicular first and second axes, the first magnetic piston and second magnetic piston are arranged in fixed relation to each other along the first and second axes, and a magnetic piston of a link received within a coil of an adjacent link; and
(ii) an electrically insulating biologically inert membrane covering at least a portion of a surface of the mesh.

43. The device of claim 42, wherein each piston includes means for carrying current and transferring current from a link to an adjacent link.

44. The device of claim 42, wherein the carrier is generally bag-shaped with a closed end.

45. The device of claim 42, wherein the carrier is generally tubular with two open ends.

46. The device of claim 42, wherein the carrier has an opening with a fastener.

47. The device of claim 46, wherein the fastener comprises laces.
